(19) United States
(12) Patent Application Publication
(10) Pub. No.: US 2026/0354401 A1
(43) Pub. Date: Nov. 12, 2026

(54) Methods of Processing Dairy Liquids Using Gelling Agents, and Related Methods, Gelling Agents, and Products

(71) Applicant: University of Vermont and State Agricultural College, Burlington, VT (US)

(72) Inventor: Rachael Floreani, Burlington, VT (US)

(21) Appl. No.: 19/490,461

(22) PCT Filed: Jun. 5, 2024
(86) PCT No.: PCT/US2024/032652

§ 371 Date: Dec. 5, 2025

Related U.S. Application Data
(60) Provisional application No. 63/471,012, filed on Jun. 5, 2023.

(51) Int. Cl. A23C 9/154; A23J 3/08; A23J 3/22; A23L 13/00; A23L 29/256; C12N 5/00

(52) U.S. Cl. CPC A23C 9/154; A23J 3/08; A23J 3/227; A23L 13/00; A23L 29/256; C12N 5/0068; C12N 2533/50; C12N 2537/10

(57) ABSTRACT

Methods of processing dairy liquids using gelling agents, and related methods, gelling agents, and products. In some embodiments, a dairy liquid comprising water and biomolecules is processed by adding gelling agents to the diary liquid, where the gelling agents comprise net-precursor molecules selected and/or functionalized to physically capture the biomolecules in the dairy liquid, along with any needed activation agent, if any, for activating forming of a network. In some embodiments, the resulting hydrogel can be used as part of a food product or a medical biopolymer product, among other things. The resulting liquid may also be safe for disposal. Dairy producers and farmers will be able to use the disclosed methods for valorization of unused dairy liquids that result in both environmentally friendly waste that is free of the biomolecules that are captured, and hydrogels that can be used for food products.

### CROSS-REFERENCE TO RELATED APPLICATIONS

This application claims the benefit of priority of U.S. Provisional Patent Application Ser. No. 63/471,012, filed Jun. 5, 2023, and titled “Methods of Treating Dairy Liquids Using Gelling Agents,” which is incorporated herein by reference in its entirety for all purposes. International Patent Application PCT/US2023/019509, filed on Apr. 22, 2023, and titled “Polymer/Milk-Protein Hydrogels, Cell Scaffolds And Cultured Meat Products Made Therewith, And Associated Methods” in the names of Floreani et al., is also incorporated herein by reference in its entirety for all of its teachings relevant to the present disclosure.

### FIELD OF THE INVENTION

The present invention generally relates to the field of processing dairy liquids. In particular, the present invention is directed to methods of processing dairy liquids using gelling agents, and related methods, gelling agents, and products.

### BACKGROUND

Production of dairy products, such as milk, cream, butter, cheese, and yogurt, among others, generates unused dairy liquids, such as equipment wash-down water, unused liquids from cheese and butter making, and discarded milk, among others, that need to be handled in an environmentally sound manner. For example, many diary liquids are subject to strict environmental restrictions on their disposal because they contain organic matter, nutrients, and chemicals that could contaminate ground and/or surface water if not properly managed, and could also deplete the soil of nutrients.

### SUMMARY OF THE DISCLOSURE

In one implementation, the present disclosure is directed to a method of processing a dairy liquid, wherein the dairy liquid comprises water and biomolecules. The method includes adding a gelling agent to the dairy liquid to create a mixture of the gelling agent with the dairy liquid, wherein the gelling agent comprises net-precursor molecules selected and/or functionalized to physically capture the biomolecules in the dairy liquid; causing the net-precursor molecules to conjugate with one another within the mixture so as to form a hydrogel containing the biomolecules and a molecular net formed from the net-precursor molecules; and separating the hydrogel from the water so as to clarify the dairy liquid.

In another implementation, the present disclosure is directed to method of making a food product. The method includes receiving a dairy liquid that contains water and biomolecules; adding a gelling agent to the dairy liquid to create a mixture of the gelling agent with the dairy liquid, wherein the gelling agent comprises net-precursor molecules selected and/or functionalized to physically capture the biomolecules in the dairy liquid; causing the net-precursor molecules to conjugate with one another within the mixture so as to form a hydrogel containing the biomolecules and a molecular net formed from the net-precursor molecules; and separating the hydrogel from the water as the food product.

In yet another implementation, the present disclosure is directed to a gelling agent for forming a hydrogel containing biomolecules present in an aqueous mixture. The gelling agent includes net-precursor molecules comprising one or more carbohydrate molecules and/or one or more protein molecules, wherein the net-precursor molecules are: modified with a crosslinking reagent that allows the net-precursor molecules to crosslink with one another in the aqueous mixture to form the hydrogel; and functionalized to physically capture the biomolecules in the hydrogel.

In still another implementation, the present disclosure is directed to a method of making a gelling agent for forming a hydrogel containing biomolecules in an aqueous mixture. The method includes providing net-precursor molecules comprising one or more carbohydrate molecules and/or one or more protein molecules; chemically modifying the net-precursor molecules with a conjugation reagent that allows the net-precursor molecules to conjugate with one another in the aqueous mixture to form the hydrogel; and chemically modifying the net-precursor molecules to physically capture the biomolecules in the hydrogel.

### BRIEF DESCRIPTION OF THE DRAWINGS

For the purpose of illustrating aspects of the disclosure, the drawings show aspects of one or more embodiments of the disclosure. However, it should be understood that the present disclosure is not limited to the precise arrangements and instrumentalities shown in the drawings.

FIG. 1 is a schematic diagram of an example process for valorizing unused dairy liquids to create comestible products and intermediates;

FIG. 2 is a representation of an alginate molecule in the presence of a divalent cation that can provide ionic bonding between the alginate molecule and other molecules within a hydrogel of the present disclosure;

FIG. 3A is a representation of the alginate molecule of FIG. 2 that has been chemically modified via methacrylation to include acrylate groups to provide covalent crosslinking through controlled radical polymerization;

FIG. 3B is a graph of each of storage modulus (G′) and loss modulus (G″) versus time for differing wavelengths of photonic stimulation for a hydrogel precursor containing the methacrylated alginate represented in FIG. 3A;

FIG. 4 is a representation of the chemically modified alginate molecule of FIG. 3A that has been further chemically modified to add aldehyde groups that provide functionality to allow additional crosslinking to primary amine groups of milk protein(s) within a hydrogel of the present disclosure;

FIG. 5A is a representation of the chemically modified alginate molecule of FIG. 4 in the presence of a hydrophobic molecule and that has been further chemically modified to add cyclodextrin for creating hydrophobic-hydrophobic conjugation with other hydrophobic molecules;

FIG. 5B is a representation of multiple alginate molecules of FIG. 5A conjugated with one another via hydrophobic-hydrophobic interactions among added cyclodextrin and hydrophobic molecules;

FIG. 5C is a representation of an interconnected polymeric network of an example hydrogel of the present disclosure, illustrating conjugations and/or crosslinking between differing polymer chains and conjugations and/or crosslinking between similar polymer chains;

FIG. 6 illustrates a process for chemically modifying alginate molecules to link cyclodextrin molecules to the alginate molecules via (CH₂)₆ and (CH₂)₂ carbon chains;

FIG. 7A is a representation of an alginate molecule chemically modified at the carboxyl site;

FIG. 7B is a representation of an alginate molecule chemically modified at the hydroxyl sites and polymer backbone;

FIG. 7C is a representation of an alginate molecule chemically modified at the hydroxyl site;

FIG. 7D is a representation of an alginate molecule chemically modified at the hydroxyl site and polymer backbone;

FIG. 7E is a representation of an alginate molecule chemically modified at the polymer backbone;

FIG. 7F is a representation of an alginate molecule chemically modified at the hydroxyl and carboxyl sites;

FIG. 8A is a representation of the chemically modified alginate molecule of FIG. 4 further chemically modified to include a cell-adhesion-promoting (CAP) functional group, here, an arginylglycylaspartic acid (RGD) functional group,

FIG. 8B is a representation of the chemically modified alginate molecule of FIG. 5A further chemically modified to includes a (CAP) functional group, here, an RGD functional group;

FIG. 9A is a photograph showing two hydrogels formed in corresponding milk wash water solutions;

FIG. 9B is a graph of transmittance versus wavelength for each of the alginate+milk wash water solution prior to adding the CaCl₂, the supernatant after adding the CaCl₂ and after the resulting ionic crosslinking, and the CaCl₂ solution;

FIG. 9C is a graph of transmittance versus wavelength for each of the modified alginate+milk wash water solution prior to adding the CaCl₂ and covalent crosslinking, the supernatant after adding the CaCl₂ after ionic and covalent crosslinking, and the CaCl₂ solution;

FIG. 10A is a photograph of whey concentrate;

FIG. 10B is a top-view photograph of microbeads formed from mixing a chemically modified alginate with whey concentrate;

FIG. 10C is a side-view photograph of microbeads formed from mixing a chemically modified alginate with whey concentrate;

FIG. 10D is a photograph of a hydrogel after it was removed from a bath of calcium chloride;

FIG. 11A is a photograph of whey concentrate;

FIG. 11B is a photograph of microbeads formed from mixing alginate with whey concentrate;

FIG. 11C is a photograph of foamed clumps that formed after microbeads from FIG. 11B spent about five minutes on a vortex;

FIG. 11D is a photograph of calcium chloride solution added directly to a solution of alginate and whey; and

FIG. 11E is a photograph of a hydrogel that formed after spending about one day crosslinking in a bath of calcium chloride.

### DETAILED DESCRIPTION

### General

Unless otherwise defined, all terms of art, notations, and other scientific terminology used herein are intended to have the meanings commonly understood by those of ordinary skill in the art. In some cases, terms with commonly understood meanings are defined herein for clarity and/or for ready reference, and the inclusion of such definitions herein should not necessarily be construed to represent a difference over what is generally understood in the art.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

As used herein, the term “about” when used with a corresponding numeric value refers to ±20% of the numeric value, typically ±10% of the numeric value, often ±5% of the numeric value, and most often ±2% of the numeric value. In some embodiments, the term “about” can mean the numeric value itself. In certain embodiments, where applicable, the term “about” indicates the designated value(s) ±one standard deviation of that/those value(s).

As used herein, the term “hydrogel” will be known to one of ordinary skill in the art and includes crosslinked polymeric material that has three-dimensional polymer networks. Hydrogels can be prepared from natural, synthetic, or synthetic/natural hybrid polymers. Biopolymer hydrogels can be formed by covalent crosslinking, chemical conjugation, esterification, and polymerization.

Methods for synthesizing hydrogels are known to those of ordinary skill in the art and include different polymerization methods using both chemical and physical crosslinking routes. For example, chemically crosslinked hydrogels can be synthesized using methods including, but not limited to, chain growth polymerization, addition and condensation polymerization, and gamma and electron beam polymerization. Additionally, physically crosslinked hydrogels can be synthesized using methods including, but not limited to, electrostatic interaction, crystallization, stereocomplex formation, hydrophobized polysaccharides, protein interaction, and hydrogen bonding; and ionically crosslinked hydrogels can be synthesized using methods including, but not limited to, ionic interaction.

Alginate-dairy byproduct biopolymer blend hydrogels have been previously investigated for several applications including dairy wastewater treatment. However, previous work focuses on flocculating solids in suspension within the dairy wastewater and filtering-out the solids. In this disclosure and in some embodiments, the filtering of flocculated solids is circumvented by utilizing a system that uses one or more gelling agents to form a hydrogel that can then be turned into an additional revenue-generating product with little economic impact, while at the same time allowing the remaining liquid (mostly water without the harmful environmental contaminants) to be readily disposed of. For example, the hydrogel can be used as part of a food product or as a food product itself, such as scaffolding for cultured meat or as a nutritious food product (e.g., with or without additional processing) or a medical biopolymer product, among other things. In this connection, International Patent Application PCT/US2023/019509, filed on Apr. 22, 2023, and titled “Polymer/Milk-Protein Hydrogels, Cell Scaffolds And Cultured Meat Products Made Therewith, And Associated Methods” in the names of Floreani et al. is incorporated by reference herein for all of its teachings relevant to the present disclosure including, but not limited to, example polymer molecules for the net-precursor molecules, example chemical modifications for promoting net formation and for promoting gel formation, example conjugation and crosslinking chemistries, and example uses of hydrogels formed in accordance with this disclosure, among others.

Unused dairy liquids, which are full of proteins, fats, sugars (i.e., “biomolecules”), and salts, are normally broken down enzymatically or via bacterial fermentation, which is time-consuming. In embodiments of the present disclosure, physical interaction is created between net-precursor molecules (e.g., biopolymer molecules, such as polysaccharide molecules (alginate, as an example)) of the gelling agent(s), and/or bio-nets formed thereof, on the one hand, and the target biomolecules (e.g., the proteins, fats, sugars, and/or salts alone or in any combination or subcombination) in the unused dairy liquid on the other hand to form hydrogels, or other biopolymer-based product(s), which can be deployed in any one or more of the various applications mentioned above, among others.

By creating a solution of the gelling agent(s) with unused dairy liquid, when conjugation and/or physical interaction occurs among the net-precursor molecules and the target biomolecules in the solution, a hydrogel forms and drops out of solution-all while taking less time than conventional treatments and generating a value stream. This leaves clear water that indicates the hydrogel formation pulls out biological material and the water can then be disposed of in an environmentally and commercially viable manner. The hydrogel is a combination of the conjugation (e.g., crosslinking) of the net-precursor molecules (that will form a molecular net) with one another and physical interactions between net-precursor molecules and the target biomolecules initially suspended in the dairy liquid.

There is efficacy in methodologies disclosed herein as solutions to the very large problem of waste in the dairy industry worldwide. This technology can be used effectively and can be beneficial worldwide. Alginate, specifically, and the divalent cation (here, Ca²⁺) used for ionic conjugation in some of the examples are not the only materials that will work as gelling agents. Generally, any net-precursor molecules (e.g., biopolymer molecules such as carbohydrate molecules (e.g., polysaccharide molecules) and/or protein molecules, or, more generally, any combination of monomer molecules, oligomer molecules, and polymer molecules, singly or in any combination or subcombination) that, under proper conditions and/or properly modified and in the presence of any one or more activation agents (e.g., divalent cations, crosslinking agent(s), initiator(s), etc.) that work together with the net-precursor molecules to form a molecular net and capture the suspended biopolymers and condense the materials (after conjugation, crosslinking, and/or physical interaction) into a hydrogel that can be used. Various examples of each are disclosed herein, such as in the appended drawings and the appended claims.

Embodiments of the present disclosure can be used to create one or more useful products, valorize dairy liquids, support a food system for an increasing global population, and/or provide medical biopolymers, among other things. The hydrogel-based products of the present disclosure are not limited to the applications mentioned, as the methodologies of the present disclosure represent a broad platform from which different formulations and functions can be created and implemented. Both the water treatment and food product generation are particularly equally attractive from a novel system generation viewpoint, with medical biopolymer formation also being attractive. In this connection, it is noted that while many of the examples are directed to valorizing wastewater, the same or similar methodologies can be used with other dairy liquids, such as non-wastewater containing the target biomolecules, such as dairy by-product taken from off of a cheese-processing line or other diary processing line.

Embodiments of the present disclosure can be integrated into the water collection process from dairy food producers and dairy farmers. Preliminary data shows proof of concept of the material as both producing a product and helping to eliminate waste-dairy liquid of biomolecules for safe disposal. As those of ordinary skill in the art will readily appreciate, all of the formulations made so far can be modified and optimized (tailored), including the chemical makeup of the material used to condense biopolymers in dairy liquids.

As discussed above, a major problem of the dairy industry is the disposal of milk (low grade, contaminated, or otherwise), water use, and wastewater generation. For example, if wastewater can be treated effectively, then water use can be reduced and the negative environmental effects can decrease. The problem is global, and sustainable solutions are still being sought, even within the largest companies and distributors. Also, problems with prohibitive cost can be offset by valorization of the trapped biological components in dairy wastewater. Technology disclosed herein uses both a physical interaction between the polysaccharide (i.e., alginate) and the proteins, sugars, and/or lipids suspended in the dairy wastewater, but also depends on forming a hydrogel network that further entraps the biopolymers. Important parameters include pH, ions present, concentration of biopolymers (e.g., proteins), salt concentration, and batch to batch variability, among others. The work that most investigators and companies are doing is based on fermentation or some degradation of the biopolymers in milk and milk waste of some kind. In contrast, methodologies disclosed herein do not dissolve the organic component, nor do they just flocculate the organic material and remove it for fertilizers, energy generation, or something else. Rather, the disclosed methodologies can be implemented to generate a bulk solid product that can be used for food or medical applications.

Dairy producers (such as milk, cream, cheese, yogurt, butter, etc., producers) can use a disclosed methodology to remove biomolecules (e.g., biopolymers and lipids) from, for example, the wastewater they use to flush/clean the milk production line. Dairy farmers will be able to use a disclosed methodology for valorization of low-grade and/or waste milk and/or wash-water used to clean milking and milk-storage equipment. In both scenarios, customers will be able to use disclosed methodologies to generate value from waste. And in particular, the gel-based product that collected using a disclosed methodology can be used to create food products with high nutritional and protein content and biopolymer-based medical products.

### Polymer(s)

As used herein, a polymer of a hydrogel of the present disclosure may be any polymer that is suitable for forming the desired interconnecting polymer network of the hydrogel and is compatible with the application at issue. Regarding the latter, for example, for a hydrogel used to create a comestible, each polymer may need to be a food-grade polymer, for example, as approved by an appropriate authority, such as the Food and Drug Administration in the U.S. In some embodiments, a polymer may be a biopolymer such as, but not limited to, a plant-based biopolymer, such as, but not limited to, starches, celluloses, pectins, glycogens, and polysaccharides. In some embodiments, a polysaccharide may be a naturally occurring polysaccharide, such as, for example, hyaluronan, dextran, chitosan, chondroitin, alginate, or agarose, among others. In some embodiments, a plant-based polysaccharide may be an alginate. It is noted that as referred to herein and in the appended claims, any biopolymer noted can be in any effective state, such as a native state or a modified state, for example, a genetically modified state or a denatured state, among others, unless specifically indicated otherwise.

A polymer of a hydrogel of the present disclosure may be homopolymer, a heteropolymer, or a polymer blend, such as a homopolymer-copolymer blend, among others. When multiple polymers are present in a hydrogel of the present disclosure, only one, fewer than all, or all of the polymers may be chemically modified in one or more differing ways for producing the conjugation(s) desired interconnecting the polymer network of the hydrogel. In some embodiments, the polymer(s), for example any one or more of the polymers mentioned above or other suitable polymer(s), may be present in an amount, either in a weight percentage or a volume percentage of the hydrogel (hydrated or dehydrated), of about 0.001% to about 99.999%, of about 0.01% to about 80%, of about 0.01% to about 50%, of about 0.1% to about 25%, among other ranges.

### Milk Protein(s)

As used herein, a milk protein of a hydrogel of the present disclosure refers to proteins or protein equivalents and/or variants that can be obtained from the natural milk of any suitable animal, such as a dairy cow, a goat, a sheep, a buffalo, a camel, or a yak, among others. Natural milk includes water, lactose, proteins, lipids, and minerals, among other things, including pigments, enzymes, and trace amounts of gases. It is noted that while a hydrogel of the present disclosure includes one or more milk proteins, in some embodiments the hydrogel may include one or more, or none, of the other components of the milk as may be desired for a particular hydrogel composition. For a hydrogel of the present disclosure intended for cultivating living cells, the milk protein(s) may be selected based on its/their cell-adhesion characteristic(s) and/or cell-proliferation characteristic(s). In some embodiments, milk protein(s) can replace an expensive cell adhesion ligand, specifically, an arginylglycylaspartic acid (RGD) peptide motif, and/or other animal components such as collagen and fibronectin. In addition and as discussed below, a milk protein of the present disclosure may be modified to enhance its cell-adhesion ability.

Milk proteins generally fall into one or the other of two families, namely, casein and whey. The casein family proteins generally consist of several types (αs1, αs2, β, and κ), with a mix of α-s1 and α-s2 (i.e., α-casein) predominating. The whey family proteins generally consist of β-lactoglobulin, α-lactalbumin, blood serum albumin, immunoglobulins, lactoferrin, transferrin, and a variety of minor proteins and enzymes, with β-lactoglobulin predominating. In some embodiments of a hydrogel of the present disclosure, whey protein(s) can be preferred. In some embodiments of a hydrogel of the present disclosure, β-lactoglobulin can be preferred because of its naturally good cell-adhesion properties. When whey protein is used in a hydrogel of the present disclosure, the whey protein may be provided in any suitable form, such as, for example, part of a whey protein isolate (WPI) or part of a whey-protein concentrate, among others. In some embodiments, using whey protein may be desirable when they are a residual waste product of other processes and thus allow for a sustainable source and means to reduce current industrial waste.

As with a polymer used in creating a hydrogel of the present disclosure, it is noted that as referred to herein and in the appended claims, any milk protein noted can be in any effective state, such as a natural state or a modified state, such as a genetically modified state (e.g., recombinant) or a denatured state, among others, unless specifically indicated otherwise. When multiple milk proteins are present in a hydrogel of the present disclosure, the multiple proteins may be of differing types from the same animal (e.g., multiple whey proteins, whey protein(s) and casein), may be of the same type(s) from differing animals, or both of differing types from the milk of the same animal and of the same type(s) from differing animals. Fundamentally, there are no limitations on the milk protein(s) that can be used in creating a hydrogel of the present disclosure. In some embodiments, the milk protein(s), for example any one or more of the milk proteins mentioned above, may be present in an amount, either in a weight percentage or a volume percentage of the hydrogel (hydrated or dehydrated), in an amount of about 0.001% to about 99.999%, of about 0.001% to about 80%, of about 5% to about 50%, of about 10% to about 30%, among other ranges.

### Chemical Modification(s)

As used herein, chemical modifications pertinent to the formation of a hydrogel of the present disclosure include (1) chemical modifications that can be used singly or in various combinations with one another to cause or promote formation of an interconnecting polymer network within a hydrogel of the present disclosure, and (2) chemical modifications that can be used to cause or promote the adhesion of living cells to the interconnecting polymer network. Examples of each of these chemical modifications are discussed below.

Chemical Modifications for Forming Polymer Network. Each polymer and/or each milk protein may be chemically modified using one or more chemistries to create or promote conjugation(s) of the molecules within a hydrogel of the present disclosure so as to form an interconnected polymer network that comprises the molecules, for example, to form an extracellular matrix (ECM) for cultivating living cells. Depending on the type(s) of chemical modification(s), the conjugation(s) may be of any suitable type, such as ionic crosslinking, covalent crosslinking through controlled radical polymerization (e.g., of the polymer(s)), reversible covalent crosslinks (e.g., reactions of primary amines within the milk protein(s)), and hydrophobic-hydrophobic interactions (e.g., via cyclodextrin bonding onto one or more types of polymers, (e.g., polysaccharides), among others. Examples of these chemical modifications are described below and illustrated in the accompanying FIGS. 3A through 8B in the context of the polymer being alginate. Those of ordinary skill in the art will readily appreciate that while these examples involve alginate, the same or similar chemistries can be used for other biopolymers and polymers. As mentioned above, each of these chemical modifications can be used singly, in combination with one another, or in any subcombination with one another.

The appended example claims are incorporated in this section by reference so as to be considered to form part of this Detailed Description section as if originally presented herein. These claims disclose example methods and products and related aspects of each. Those having ordinary skill in the art will be able to practice the subject matter of these claims and any variation apparent thereto based on the teachings of this disclosure using only routine knowledge in the art and without undue experimentation. Where generic terminology is used and only limited species examples are provided, those of ordinary skill in the appropriate art will be able to substitute the disclosed species with other species known in the art to be equivalent to the disclose species and/or provide the same or similar functionality(ies) as the disclosed species.

Aspects of the present disclosure will now be described in more detail with reference to the accompanying drawings. The accompanying drawings show exemplary embodiments of various aspects of the present disclosure. These and other aspects may, however, be embodied in many different forms and should not be construed as limited to the embodiments set forth herein.

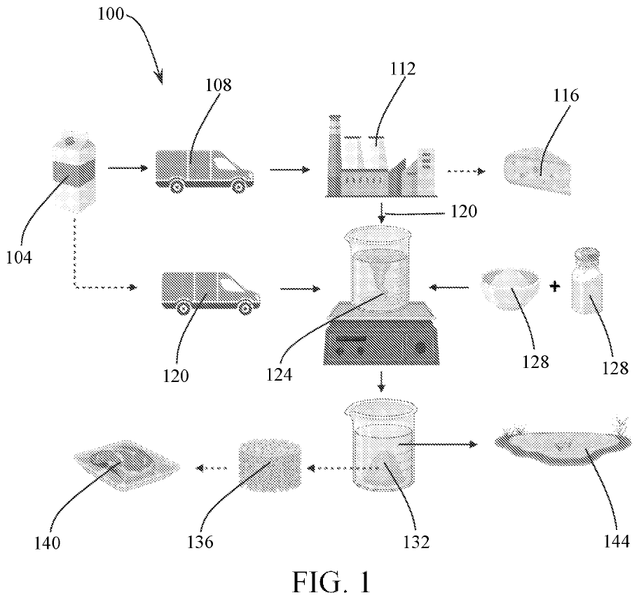

FIG. 1 is a schematic diagram of an example process 100 for valorizing dairy liquid to create comestible products and intermediates. In this diagram, the milk source 104 can be a suitable source of milk, such as a dairy farm and/or any milk aggregator, such as a dairy coop or regional supplier, among others. Usable milk is transported 108 to a processing facility 112, wherein the usable milk is processed into any one or more dairy products 116, such as, but not limited to, cheese(s), packaged milk(s), cream(s), butter(s), yogurt(s), sour cream, buttermilk, ice cream, etc.

Unused dairy liquid 120 from each of the milk source 104 and processing facility 112 is provided for valorization 124, which involves processing the unused dairy liquid 120 with one or more gelling agents 128 that produce a hydrogel 132 containing biomolecules and other molecules from the unused dairy liquid 120. Examples of unused dairy liquid 120 suitable for such valorization 124 include wash water from dairy-production equipment, unused liquids from cheesemaking, butter-making, and yogurt-making, unused liquids from milk and/or cream processing, and discarded milk, among others. The unused dairy liquid 120 may comprise water and biomolecules, which may comprise at least one protein, lipid, and/or sugar. Examples of protein include, but are not limited to, whey and/or casein. The one or more gelling agents 128 may comprise net-precursor molecules selected and/or functionalized to physically capture the biomolecules in the dairy liquid, along with any needed activation agent, if any, for activating forming of a network. For example, gelling agents 128 may comprise alginate, which is one example of a net-precursor molecule, and a salt, which is one example of an activation agent. When the gelling agent(s) is/are mixed with the unused dairy liquid 120, the net-precursor molecules crosslink with one another to form the hydrogel 132 and capture biomolecules in the hydrogel 132.

The hydrogel 132 may then be used to create scaffolding 136, which may be a precursor to a finished food product 140. For example, scaffolding 136 may be seeded with one or more types of living cells that are proliferated so as to produce one or more food products 140, such as one or more cultured meat products. Various types of protein cells and/or lipid cells may be cultured within the scaffolding 136 so as to make the food product 140. A byproduct of the valorization 124 is environmentally friendly waste 144 that can be readily disposed of, unlike the unused dairy liquid 120 prior to valorization 124.

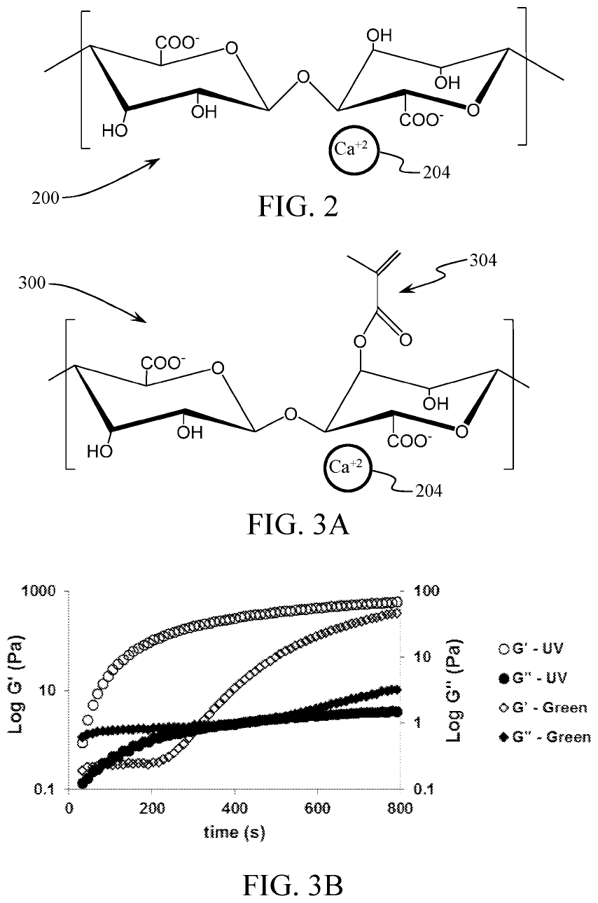

As set forth above, gelling agents 128 may comprise net-precursor molecules selected and/or functionalized to physically capture biomolecules in dairy liquid 120, along with any needed activation agent, if any, for activating forming of a network. FIGS. 2 through 8B show example polymer molecules for the net-precursor molecules. Net-precursor molecules may comprise biopolymer molecules, protein molecules, and/or carbohydrate molecules, including, but not limited to, polysaccharide molecules, such as alginate molecules. In some embodiments one or more activation agents may also be added. For example, a salt, such as a calcium-based salt, may be added to facilitate conjugating of the net-precursor molecules with one another and to facilitate bonding of the biomolecules to the net-precursor molecules. The net-precursor molecules may also be functionalized or acrylated to facilitate the crosslinking of the net-precursor molecules with one another. For example, the net-precursor molecules may be functionalized with a photoreactive crosslinking reagent. The net-precursor molecules may also be functionalized to facilitate the net-precursor molecules physically capturing the biomolecules, to facilitate ionic bonding of the net-precursor molecules with the biomolecules, to facilitate covalent crosslinking of the net-precursor molecules with the biomolecules, to facilitate secondary crosslinking of the net-precursor molecules with the biomolecules, and/or to facilitate cell adhesion. Compounds that may be used to functionalize the net-precursor molecules include, but are not limited to, an aldehyde, a cyclodextrin, poly(propylene glycol), poly(ethylene glycol), and an RGD peptide. Applying an external crosslinking stimulus, such as light, or adding one or more crosslinking reagents, such as a homobifunctional reagent or a heterobifunctional reagent, may also facilitate causing the net-precursor molecules to crosslink with one another.

FIG. 2 shows an example of a “backbone” alginate molecule 200 in the presence of a divalent cation 204 (here, a divalent calcium cation) that can create divalent cation-initiated crosslinking with another alginate molecule (not shown). Cations other than calcium cations, including other divalent cations, can be additionally or alternatively used.

FIG. 3A shows an example of a chemically modified backbone alginate molecule 300 chemically modified via an acrylation chemistry to provide the modified alginate molecule with, for example, methacrylate groups 304 (here, via methacrylic anhydride (MA)) to allow covalent crosslinking through controlled radical polymerization, for example, via photonic stimulation. In FIG. 3A, the covalent crosslinking via the methacrylate groups 304 provides a dual-crosslinking with the divalent ionic crosslinking via the cation 204 of FIG. 2. In this example, the divalent cation 204 is also present. However, in other embodiments, the divalent cation 204 is not present. FIG. 3B illustrates the storage modulus (G′) and loss modulus (G″) versus time for ultraviolet light (UV) and green light induced crosslinking of the methyl acrylate modified alginate molecule 300 of FIG. 3A.

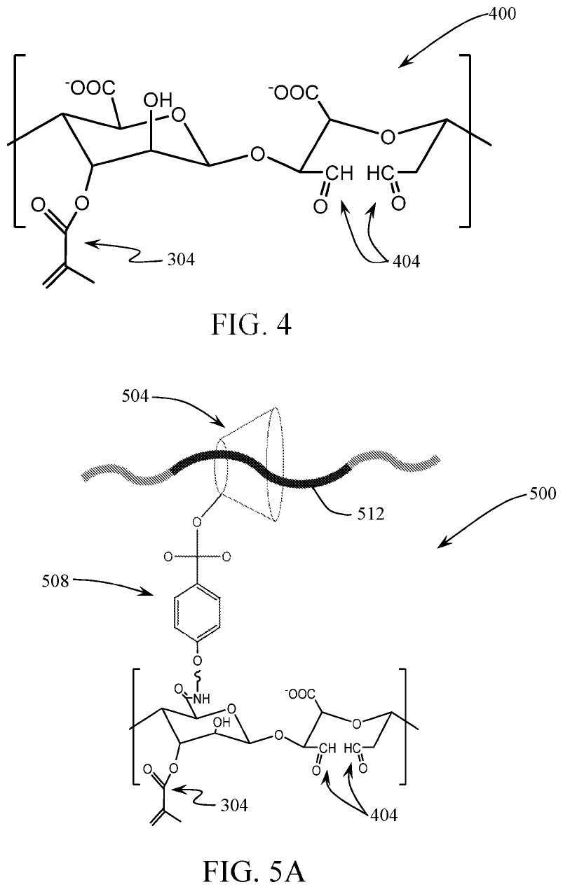

FIG. 4 shows an example of a chemically modified backbone alginate molecule 400 that is similar to the chemically modified alginate molecule 300 of FIG. 3A but is further chemically modified to include, in addition to the acrylate groups 304, aldehyde groups 404 using a suitable chemistry. In an example reaction, sodium periodate is used to oxidize the alginate uronic ring, exposing the two aldehydes. Other reactions may be used to expose the aldehydes. In this example, the aldehyde groups 404 provides covalent crosslinking to primary amines (not shown) of the milk protein(s) (not shown) in an overall interconnected polymer network, for example, an ECM. In other embodiments, this same chemistry can be used without the methacrylation chemistry for forming a crosslinked hydrogel comprising alginate dialdehyde and milk protein (not shown).

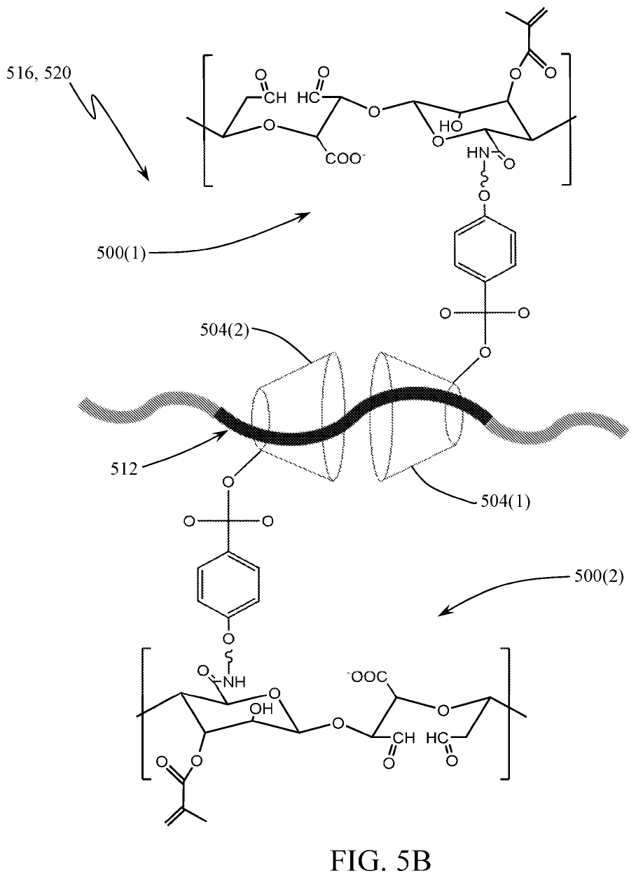

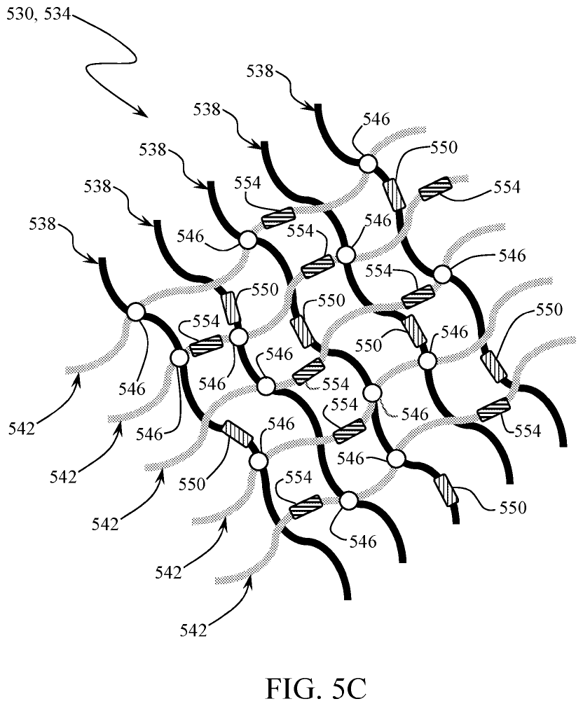

FIG. 5A shows an example of a chemically modified alginate molecule 500 that is similar to the chemically modified alginate molecule 400 of FIG. 4 that includes the acrylate groups 304 and the aldehyde groups 404 but is further chemically modified to include cyclodextrin (CD) 504 using a suitable chemistry. In this example, the CD 504 is linked to the backbone of the modified alginate molecule via polyethylene glycol (PEG) 508. In some embodiments, the CD 504 can serve a dual role. It can entrap milk protein 512 via hydrophobic-hydrophobic interactions as shown, or in the presence of a tri-block copolymer comprising two hydrophilic regions and a hydrophobic region it can form a self-healing hydrogel based on similar hydrophobic-hydrophobic interaction. In this example, the milk protein 512 forms a physical bond with the CD 504 such that pairs (not shown, but see FIG. 5B) of alginate molecules 504 that each have the CD 504 modification can conjugate with one another via the hydrophobic-hydrophobic interactions with one or more suitable hydrophobic molecules, such as the milk protein 512 of this example. Those of ordinary skill in the art will readily appreciate that molecules other than CD having hydrophobic affinity can be used, as well as that linking agents other than PEG can be used in place of or in addition to the PEG. The example alginate molecule 500 of FIG. 5A is light-responsive, capable of multiple crosslinking methods, and self-healing, and can form reversible physical and covalent interaction.

FIG. 5B illustrates an example of a conjugation of a plurality of chemically modified alginate molecules, here, two chemically modified alginate molecules 500(1) and 500(2) each being the same as the chemically modified alginate molecule 500 of FIG. 5A. In this example, the chemically modified alginate molecules 500(1) and 500(2) are conjugated with one another via their respective CD 504(1) and 504(2) via the hydrophobic-hydrophobic interactions of the CD 504(1) and 504(2) with the milk protein 512. As can be appreciated, this conjugation partially forms an interconnected polymeric network 516 of a CD-modified alginate (Alg-CD)/milk protein hydrogel 520 of the present disclosure.

Expanding on the example interconnecting network 516 of the hydrogel of FIG. 5B, FIG. 5C illustrates a broader example of an interconnected polymeric network 530 of an example hydrogel 534 of the present disclosure. In the example of FIG. 5C, the interconnected polymeric network 530 comprises first polymer chains 538 and second polymer chains 542 that are conjugated with one another via heteroconjugations 546. Within individual ones of the first polymer chains 538 and individual ones of the second polymer chains 542, the individual polymers (not individually labeled to avoid cluttering the figure) are conjugated with one another, respectively, via homoconjugations 550 and 554. As will be appreciated from reading this entire disclosure, each of the heteroconjugations 546 and each of the homoconjugations 550 and 554 may be any suitable type of conjugation, including, but not limited to, ionic crosslinking, covalent crosslinking through controlled radical polymerization, secondary crosslinking, and hydrophobic-hydrophobic interactions, among others.

FIG. 6 illustrates an example process 600 functionalizing alginate 604 with CD 608 to create two chemically modified alginates 612(1) and 612(2) having hydrocarbon chains 616(1) and 616(2), respectively, of differing lengths. In this example, the hydrocarbon chain 616(1) is (CH₂)₆ and the hydrocarbon chain 616(2) is (CH₂)₂. In FIG. 6, the following apply: “Alg” is alginate; “C6” is (CH₂)₆, “C2” is (CH₂)₂; “TosCl” is 4-toluenesulfonyl chloride; “NaOH” is sodium hydroxide; “RT” is room temperature; “min” is minute(s); “equiv” is equivalent(s); “HDA” is 1,6-hexanediamine; “DMF” is dimethylformamide; “EDA” is ethylene diamine; “h” is hour(s); “BOP” is (benzotriazol-1-yloxy)tris(dimethylamino) phosphonium hexafluorophosphate; “DMSO” is dimethyl sulfoxide; “NHS” is N-hydroxysuccinimide; and “EDC” is 1-ethyl-3-(3dimethylaminopropyl)carbodiimide. As those of ordinary skill in the art will readily appreciate, the process 600 illustrated in FIG. 6 is merely exemplary and non-limiting.

As noted, the above chemical modifications are merely examples of the types of chemical modifications that can be used to chemically modify the polymer(s) and/or the milk protein(s) to create a hydrogel of the present disclosure. A partial list of suitable chemical modifications to create or promote covalent bonding, secondary bonding, and/or hydrophobic-hydrophobic interactions includes, but is not limited to acrylation, methacrylation, oxidation, carbodiimide modification, diamine modification, dihydrazide modification, esterification, acetylation, phosphorylation, sulfation, alkylation, ethylation, arylation, amination, amide modification, pegylation, graft copolymerization, and aldehyde modification (monoaldehyde or polyaldehyde), among others. While alginate is used as the backbone polymer, those of ordinary skill in the art will understand that the example chemical modifications can be applied to other polymers, such as other polysaccharides and other biopolymers more generally. A partial list of biopolymers that can be modified using the example chemical modification includes, but is not limited to, polysaccharides (e.g., alginate, agarose, chitosan, chitin, gellan gum, gum arabic, carrageenan, cellulose, carboxymethylcellulose, methyl cellulose, xanthan gum, dextran, dextran sulfate, Hyaluronan, heparin, heparin sulfate, chondroitin sulfate, dermatan sulfate, keratin sulfate, carob gum, pullulan, starches, pectins, glycogens), shorter saccharides (e.g., saccharides, disaccharides, trisaccharide, ogliosaccharides), and polymeric proteins (e.g., fibrin, collagen, fibronectin, laminin, gelatin, elastin), among others. For alginate, in particular and in some embodiments, there are generally three major groups of chemical modifications that can be performed based on the available reactive groups, but these and combinations thereof may change depending on polymer choice. These are hydroxyl modification (including methacrylation), carboxyl modification (including cyclodextrin and RGD chemistries), and modification of the polymer backbone (including oxidation).

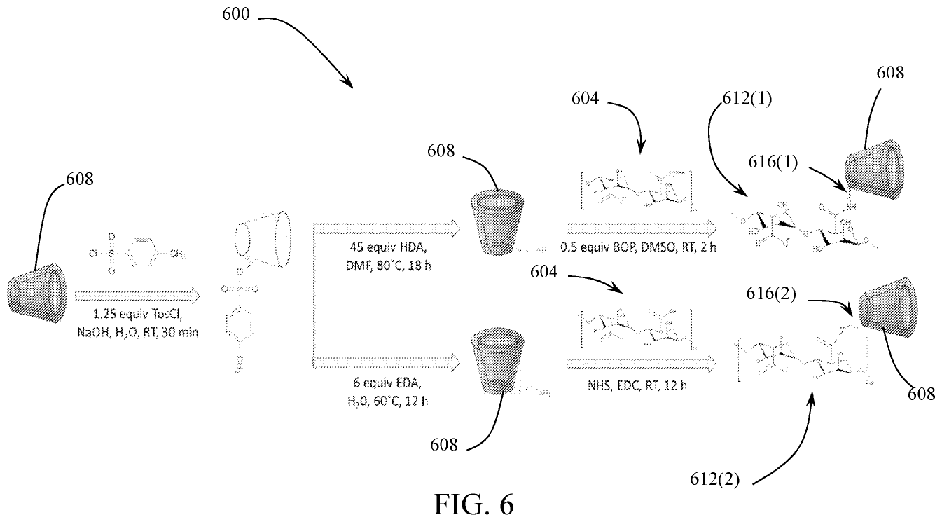

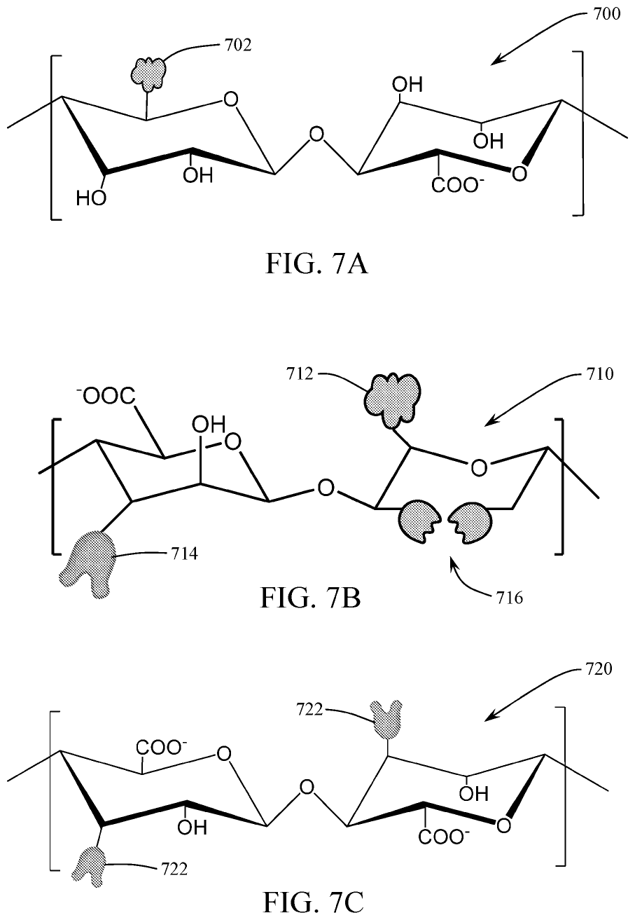

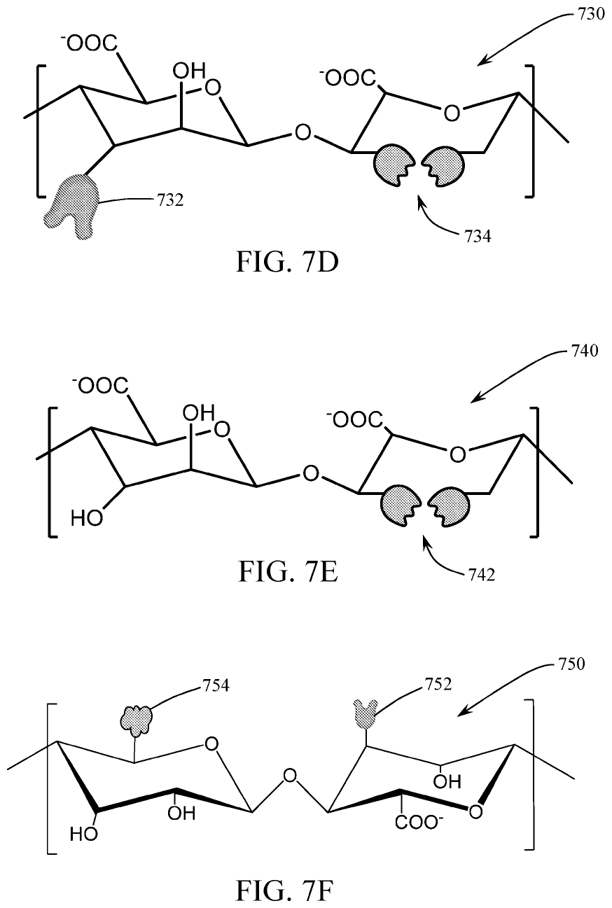

FIGS. 7A through 7F illustrate some example modifications that can be made to a polymer that is used in a hydrogel of the present disclosure. It is noted that all of these examples are based on alginate as the polymer. However, those of ordinary skill in the art will readily appreciate that similar modifications can be made to other polymers and that these chemical modifications are merely examples. FIG. 7A shows a chemically modified polymer molecule 700 that has a chemical modification 702, such as a cyclodextrin modification, present at the carboxyl site of the polymer molecule. FIG. 7B shows a chemically modified polymer molecule 710 that has chemical modifications 712, 714, and 716, present, respectively, at the hydroxyl sites and polymer backbone of the polymer molecule. For example, the chemical modification 712 at the first hydroxyl site may be, for example, a methacrylation modification; the chemical modification 714 at the second hydroxyl site may be, for example, a cyclodextrin modification; and the chemical modification 716 at the polymer backbone may be, for example, a dialdehyde modification. FIG. 7C shows a chemically modified polymer 720 that has a chemical modification 722, such as a methacrylation modification, at the hydroxyl site of the polymer molecule.

FIG. 7D shows a chemically modified polymer molecule 730 that has chemical modifications 732 and 734, present, respectively, at the hydroxyl site and polymer backbone of the polymer molecule. For example, the chemical modification 732 at the hydroxyl site may be, for example, a methacrylation modification and the chemical modification 734 at the polymer backbone may be, for example, a dialdehyde modification. FIG. 7E shows a chemically modified polymer 740 that has a chemical modification 742, such as a dialdehyde modification, at the polymer backbone of the polymer molecule. FIG. 7F shows a chemically modified polymer molecule 750 that has chemical modifications 752 and 754, present, respectively, at the hydroxyl site and carboxyl site of the polymer molecule. For example, the chemical modification 752 at the hydroxyl site may be, for example, a methacrylation modification and the chemical modification 754 at the carboxyl site may be, for example, a cyclodextrin modification.

In addition, while light can be used as the stimulus for polymerization, other types of stimuli can be used with the appropriate modification to the relevant chemistry. For example, other stimuli other than photonic stimulation can include, but are not limited to, the addition of chemical compounds, electric current, and/or magnetic fields or changes to temperature, strain, pressure, humidity, and/or pH. For each of these stimuli, each may be controlled in a manner that controls the corresponding polymerization. For example, if polymerization is stimulated at or above a polymerization temperature, Tp, then components for making a hydrogel can be mixed at a temperature lower than Tp and then the temperature of the mixture raised to equal to or greater than Tp when polymerization is desired. In this example, heat may be added in any suitable manner known in the art. As another example, if polymerization is stimulated at or below a polymerization pH, pHp, then components for making a hydrogel can be mixed at a pH higher than pHp and then the pH of the mixture lowered to equal to or less than pHp when polymerization is desired. For polymerization occurring as a function of pH, a pH adjuster can be added to the mixture at a desired/necessary time to adjust the pH of the mixture accordingly.

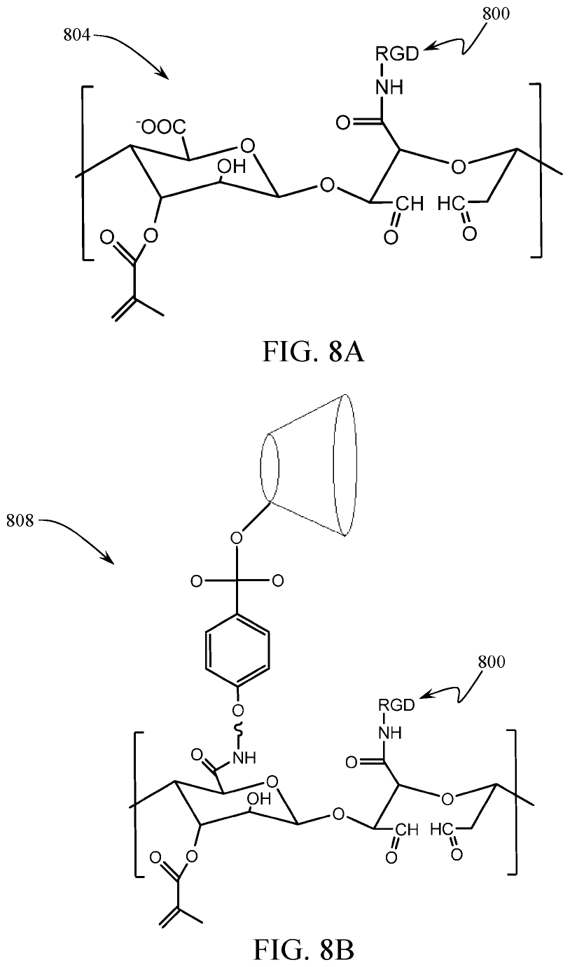

Chemical Modifications for Promoting Cell Adhesion: In embodiments of hydrogels made in accordance with the present disclosure made to support live-cell cultivation, the extracellular matrix, or cell scaffold, may be chemically modified to promote adhesion of the living cells to the scaffold. For example, molecules of the milk protein(s) and/or molecules of the polymer(s) can be chemically modified by adding one or more cell-adhesion-promoting (CAP) functional groups. In some embodiments, such functional group(s) can be covalently bonded to molecules of the milk protein(s) and/or to molecules of the polymer(s) using any suitable type of bond formation. In the context of covalent bonding of CAP functional groups, crosslinking may be performed via, for example, carbodiimide crosslinker chemistry, disulfide bond formation, or esterification, among others. Examples of CAP molecules that can be used to form the CAP functional groups that attach to the cell scaffold include, but are not limited to, arginylglycylaspartic acid (RGD) molecules, collagen molecules, fibronectin molecules, laminin molecules, and positively charged functional monomer molecules, polymer molecules, and peptides, among others. FIGS. 8A and 8B show, respectively, the chemically modified alginate molecules 400 and 500 of FIGS. 4 and 5A each further chemically modified to include a CAP functional group, here, an RGD functional group 800, so as to create corresponding functionalized chemically modified alginate molecules 804 and 808. Those of ordinary skill in the art will understand that CAP functional groups can be added to hydrogels of the present disclosure using conventional methods adopted to the relevant chemistry(ies) of the hydrogel using only knowledge known in the art.

### Cell Scaffolds (ECMs)

As noted above, a hydrogel of the present disclosure can be used to provide or make cell scaffolds, also referred to herein as ECMs, for growing and proliferating one or more types of living cells, such as to make food products such as cultured meat, to repair or replace human tissue, or to create tissue samples for testing, among other things. Using one or more of the above-identified chemical modifications and/or any other suitable modifications, properties of a cell scaffold of the present disclosure can be tuned for specific applications by altering components through additional chemical modification and/or altering the process of forming the hydrogel, for example by selecting one or more chemistries compatible with the application of the cell scaffold. Such modifiable properties include degradation, mechanical properties, swelling, and porosity, among others. Altering or tuning the properties of the material can allow for a more diverse range of cell types, including, as noted above, cells of differing meats, e.g., beef, poultry, fish, pork, etc., and differing tissue types, e.g., muscle and fat. Tuning these properties can allow more accurate imitation of complex matrices found in nature. Those of ordinary skill in the art will be able to select materials and chemistries suitable for making a hydrogel suitable for a cell scaffold of the present disclosure using knowledge common in the art and using this disclosure as a guide. Similarly, those of ordinary skill in the art will be able to tune the parameter(s) of the relevant chemistry(ies) and other processes of forming a cell scaffold of the present disclosure without undue experimentation to arrive at a cell scaffold suitable for the particular application at issue.

A cell scaffold of the present disclosure comprises a 3D structure composed of a hydrogel made in accordance with the present disclosure, such as described above and provided in examples below, and having voids or space to receive or otherwise contain seed living cells and to provide space for the seed cells to proliferate and grow so as to create the desired end product, such as cultivated meat, among other things. A cell scaffold of the present disclosure can be made in any of a variety of ways, with some examples as follows. In one example, a hydrogel can be formed in a mold having the shape of the desired final product. For example, a hydrogel precursor can be placed into a mold, with or without seed cells, and then polymerization can be initiated to create the 3D structure. The 3D structure and the mold can then be separated from one another. In some embodiments, the resulting 3D structure can be dried before seeding with living cells. In a generally related embodiment, a large mass of the hydrogel can be formed, perhaps in a mold, and then one or more 3D shapes can be cut from the large mass to create the 3D structures having the desired shape(s).

In another example, voids within the hydrogel and/or cell scaffold can be created and/or enhanced using a porogen. For example, a porogen can be added to a hydrogel precursor and the hydrogel precursor polymerized to form the hydrogel, i.e., the interconnecting polymer network. The porogen can then be removed from the hydrogel to leave the voids created and/or enhanced by the porogen. In a further example, a mass of hydrogel, hydrogel precursor, or partially crosslinked precursor can be dried (e.g., by lyophilization) and ground-up to create particles that may then be used to create a cell scaffold of a desired shape. In yet another example, the hydrogel may be formed into particles, such as spheres, either before, after full polymerization, or after partial polymerization. The particles may optionally be dried before using, and may be seeded with living cells before, during, or after formation. Each particle may be considered a cell scaffold in and of itself and fully support cell proliferation and growth. In some embodiments, a plurality of such cell-scaffold particles can be aggregated with one another, for example, within a mold, to create a larger cell scaffold having the desired 3D shape. In some embodiments, the aggregation of cell-scaffold particles may be polymerized within the mold and/or may be kept in the mold during the cell proliferation and growth process at least until the cell-scaffold particles and cells form a unitary mass able to be separated from the mold. In still further examples of forming a cell scaffold, the hydrogel, or precursor thereto (e.g., not-yet polymerized mixture) can be electro-sprayed, electrospun, applied as an emulsion, or printed using a 3D printer, layer-by-layer deposition, or any additive manufacturing, among others.

### Cultivated Meat Products

As mentioned above, a cell scaffold of the present disclosure, such as any of the cell scaffolds described in the immediately preceding subsection, any cell scaffold apparent to someone of ordinary skill in the art from that description, and any cell scaffold made using a hydrogel made in accordance with the present disclosure, can be used to create making food products, such as any of a variety of cultivated meat products, such as, for example, beef steaks, organ meat (e.g., calf liver, chicken liver, etc.), ground beef, fish fillets, lamb meat, ground chicken, chicken thigh meat, and turkey breast meat, to name just a few, and any hybrid containing meats of two or more differing species of animal.

To create a cultivated meat product of the present disclosure, a cell scaffold and/or a precursor thereto (e.g., an unformed hydrogel, a hydrogel precursor (e.g., not-yet-polymerized mixture or component(s) thereof, etc.)) is seeded with one or more types of living cells, such as myocytes, adipocytes, lipocytes, and stem cells, depending on the nature and composition of the desired cultivated meat product. For example, if fat marbling is desired in a cultivated ribeye beef steak, bovine adipocytes may be seeded into regions within a cell scaffold wherein the marbled fat is desired, while bovine myocytes are seeded into other regions wherein meat is desired.

In some embodiments and in the context of a hydrogel precursor mixture (e.g., pre-polymerization or after partial conjugation (e.g., ionic crosslinking only)), the living cells may be seeded at a density of about 10⁵ to about 10⁹ cells per milliliter of the hydrogel precursor mixture, of about 10⁵ to about 10⁸ cells per milliliter of the hydrogel precursor mixture, or of about 10⁶ to about 10⁷ cells per milliliter of the hydrogel precursor mixture, among others. In some embodiments, and in the context of a crosslinked hydrogel, the living cells may be seeded at a density of about 10⁵ to about 10⁹ cells per cubic centimeter of the hydrogel, of about 10⁵ to about 10⁸ cells per cubic centimeter of the hydrogel, or of about 10⁶ to about 10⁷ cells per cubic centimeter of the hydrogel, among others. Those of ordinary skill in the art will readily appreciate that the foregoing seeding density ranges are merely exemplary and that other seeding densities may be needed to provide optimal solutions in particular applications.

### EXAMPLES

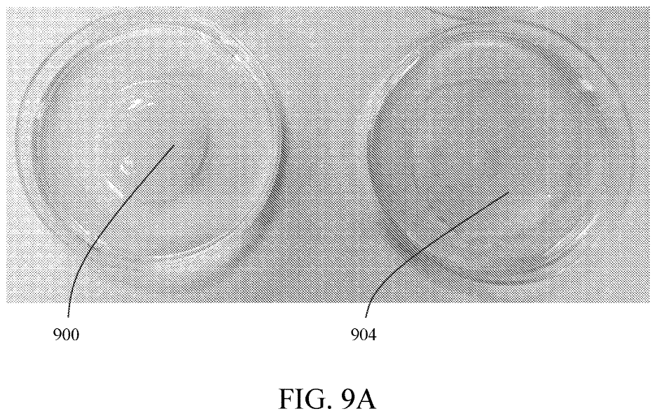

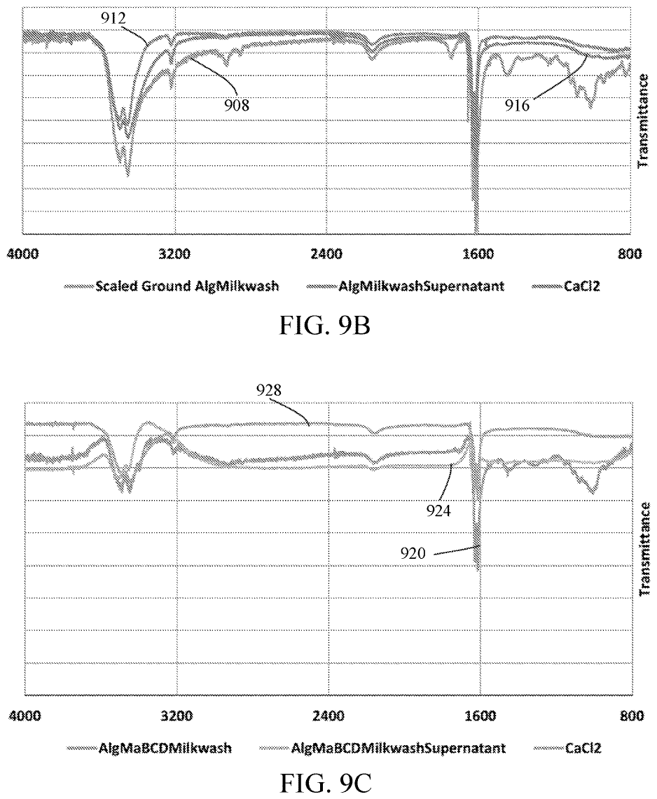

FIG. 9A is a photograph showing two hydrogels formed in corresponding milk wash water solutions. The hydrogel on the lefthand side of the photo 900 is formed via a 3% solution of alginate in milk wash water, and the hydrogel on the righthand side of the photo 904 is formed via a 3% solution of Alg-MA-B-CD in the milk wash water. Both solutions included gelling agents that comprised both net-precursor molecules and an activation agent, here about 2 mL of the net-precursor molecules (i.e., alginate or Alg-MA-B-CD) and about 3 mL of activation agent (0.5 M CaCl₂). In the hydrogel on the left 900, the backbone (molecular net) is formed by the alginate ionically crosslinking via Ca²⁺ ions. In the hydrogel on the right 904, the backbone (molecular net) is formed by the ionic crosslinking of the Ca²⁺ ions and covalent crosslinking that occurs by way of the methyl acrylate. The additional crosslinking in the hydrogel on the righthand side 904 of FIG. 9A makes that hydrogel, among other things, more robust than the ionic-crosslinked-only hydrogel on the lefthand side 900.

FIG. 9B is a graph of transmittance versus wavelength for each of the alginate+milk wash water solution prior to adding the CaCl₂ 908, the supernatant after adding the CaCl₂ and after the resulting ionic crosslinking (see FIG. 9A, lefthand side of photo 900) 912, and the CaCl₂ solution 916.

FIG. 9C is a graph of transmittance versus wavelength for each of the modified alginate +milk wash water solution prior to adding the CaCl₂ and covalent crosslinking 920, the supernatant after adding the CaCl₂ after ionic and covalent crosslinking (see FIG. 9A, righthand side of photo 904) 924, and the CaCl₂ solution 928.

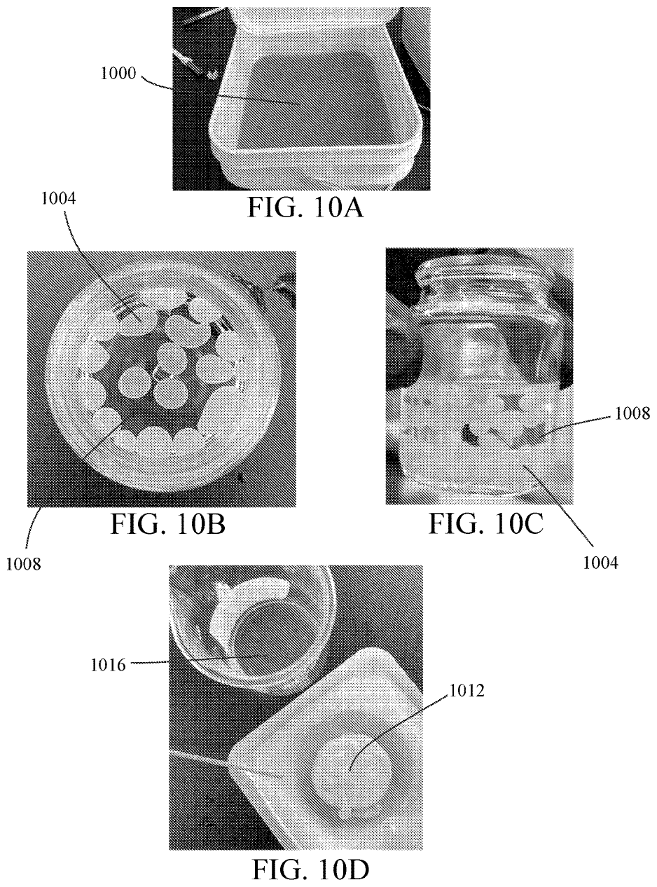

FIGS. 10A through 10D show an example of a method according to the present disclosure, where the dairy liquid 1000 is 30% whey concentrate, as shown in FIG. 10A. In this example, the gelling agents comprised a chemically modified alginate, and particularly alginate dialdehyde (AlgDa) possessing a degree of oxidation of 10%, as the net-precursor molecules and calcium chloride as an activation agent. A 4% solution was made by adding the AlgDa to the dairy liquid 1000. In one embodiment, a pipette with a cut tip was then used to transfer the AlgDa and whey solution drop wise into a bath of calcium chloride which facilitated conjugating the AlgDa molecules with one another, and forming microbeads 1004, as shown in FIG. 10B and FIG. 10C. The resulting water 1008 was clear. In another embodiment, a bulk hydrogel 1012 was also formed, by adding a calcium chloride solution 1016 directly to the AlgDa and dairy waste solution, as shown in FIG. 10D. When the AlgDa molecules conjugated with one another, they also physically captured biomolecules in the diary liquid 1000, pushing water 1008 out of the network. In this example, 20 ml of 30% whey concentrate, 0.8 g of AlgDa, and 4 ml of 2 M CaCl₂ were used.

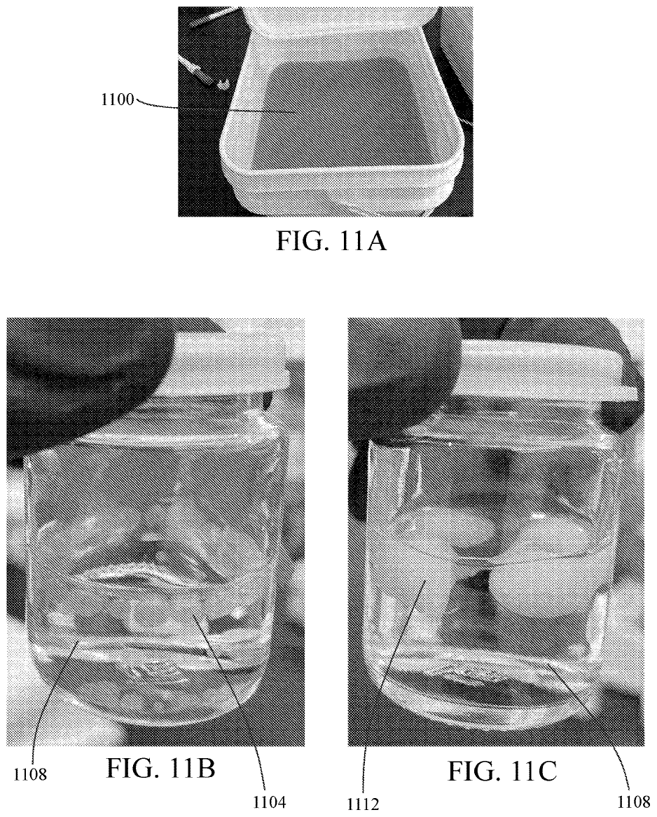

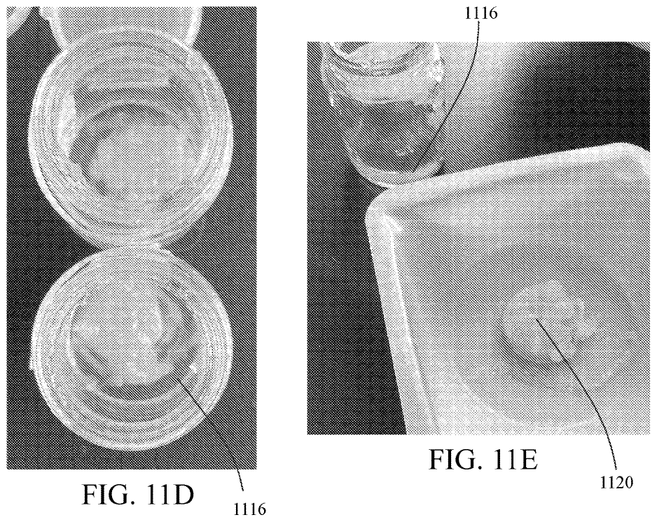

FIGS. 11A through 11E show another example of a method according to the present disclosure, where the dairy liquid 1100 is also 30% whey concentrate, as shown in FIG. 11A. In this example, the gelling agents comprised alginate as the net-precursor molecules and calcium chloride as an activation agent. A 3% solution was made by adding an alginate to the dairy liquid 1100. In one embodiment, a pipette with a cut tip was then used to transfer the alginate and whey solution drop wise into a bath of calcium chloride which facilitated conjugating the alginate molecules with one another, and forming microbeads 1104, as shown in FIG. 11B. The resulting water 1108 was clear. The foamed microbeads 1112 shown in FIG. 11C formed after microbeads 1104 spent about five minutes on a vortex. The foamed microbeads 1112 were much whiter and more aerated than the microbeads 1104, and also held their shape well. Scooping the foamed microbeads 1112 into a bath of calcium chloride facilitated conjugating the alginate molecules with one another, and a hydrogel formed. In another embodiment, a bulk hydrogel 1120 was formed by adding calcium chloride solution 1116 directly to the alginate and whey solution, as shown in FIGS. 11D and 11E. In this example, the bulk hydrogel 1120 spent about one day crosslinking in the calcium chloride solution 1116.

Various modifications and additions can be made without departing from the spirit and scope of this disclosure. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present disclosure. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this disclosure. The appended claims as originally filed with this application shall be considered to form part of this Written Description section as if contained herein upon the filing of this application.

Exemplary embodiments have been disclosed above and illustrated in the accompanying drawings. It will be understood by those of ordinary skill in the art that various changes, omissions, and additions may be made to that which is specifically disclosed herein without departing from the spirit and scope of the present disclosure.

1-138. (canceled)

139. A method of processing a dairy liquid, wherein the dairy liquid comprises water and biomolecules, the method comprising:
adding a gelling agent to the dairy liquid to create a mixture of the gelling agent with the dairy liquid, wherein the gelling agent comprises net-precursor molecules selected and/or functionalized to physically capture the biomolecules in the dairy liquid;
causing the net-precursor molecules to conjugate with one another within the mixture so as to form a hydrogel containing the biomolecules and a molecular net formed from the net-precursor molecules; and
separating the hydrogel from the water so as to clarify the dairy liquid.

140. The method of claim 139, wherein the dairy liquid comprises wash water from dairy-production equipment.

141. The method of claim 139, wherein the biomolecules comprise at least one protein.

142. The method of claim 139, wherein the net-precursor molecules comprise biopolymer molecules.

143. The method of claim 139, further comprising adding a salt to facilitate conjugating of the net-precursor molecules with one another.

144. The method of claim 139, wherein the net-precursor molecules are functionalized to facilitate crosslinking of the net-precursor molecules with one another.

145. The method of claim 139, wherein the net-precursor molecules are functionalized to facilitate the net-precursor molecules physically capturing the biomolecules.

146. The method of claim 139, wherein the net-precursor molecules are functionalized to facilitate covalent crosslinking of the net-precursor molecules with the biomolecules.

147. A method of making a food product, the method comprising:
receiving a dairy liquid that contains water and biomolecules;
adding a gelling agent to the dairy liquid to create a mixture of the gelling agent with the dairy liquid, wherein the gelling agent comprises net-precursor molecules selected and/or functionalized to physically capture the biomolecules in the dairy liquid;
causing the net-precursor molecules to conjugate with one another within the mixture so as to form a hydrogel containing the biomolecules and a molecular net formed from the net-precursor molecules; and
separating the hydrogel from the water as the food product.

148. The method of claim 147, wherein the food product comprises a scaffold for a cultured meat product.

149. The method of claim 147, further comprising culturing cells within the food product so as to make a cultured meat product.

150. The method of claim 147 wherein the dairy liquid is a waste dairy liquid.

151. The method of claim 147, wherein the biomolecules comprise at least one protein.

152. The method of claim 147, wherein causing the net-precursor molecules to crosslink with one another includes applying an external crosslinking stimulus to the mixture.

153. A gelling agent for forming a hydrogel containing biomolecules present in an aqueous mixture, the gelling agent comprising:
net-precursor molecules comprising one or more carbohydrate molecules and/or one or more protein molecules, wherein the net-precursor molecules are:
modified with a crosslinking reagent that allows the net-precursor molecules to crosslink with one another in the aqueous mixture to form the hydrogel; and
functionalized to physically capture the biomolecules in the hydrogel.

154. The gelling agent of claim 153, wherein the biomolecules comprise at least one protein.

155. The gelling agent of claim 153, wherein the net-precursor molecules are functionalized to facilitate the net-precursor molecules physically capturing the biomolecules.

156. The gelling agent of claim 155, wherein the net-precursor molecules are functionalized to facilitate covalent crosslinking of the net-precursor molecules with the biomolecules.

157. The gelling agent of claim 155, wherein causing the net-precursor molecules to crosslink with one another includes adding a crosslinking reagent to the mixture.

158. The gelling agent of claim 155, wherein the net-precursor molecules are functionalized to promote cell adhesion.
